(19) United States
(12) Patent Application Publication
(10) Pub. No.: US 2018/0325731 A1
(43) Pub. Date: Nov. 15, 2018

(54) EYE IMPLANT

(71) Applicant: RAINBOW MEDICAL LTD., Herzliya (IL)

(72) Inventor: Yossi GROSS, Moshav Mazor (IL)

(73) Assignee: RAINBOW MEDICAL LTD., Herzliya (IL)

(21) Appl. No.: 15/595,377

(22) Filed: May 15, 2017

(51) Int. Cl. A61F 9/007; A61F 9/00

(52) U.S. Cl. CPC A61F 9/00781; A61F 9/0084; A61F 9/0017

(57) ABSTRACT

Apparatus is provided for expanding a Schlemm's canal of an eye of a subject, the apparatus including an introducer having a lumen and a plurality of expandable porous structures sized and shaped to be disposed with the lumen. The expandable porous structures are configured (a) to be inserted into the Schlemm's canal at discrete locations around the canal, (b) to be inserted into the Schlemm's canal while in a collapsed form, and (c) to expand while inside the Schlemm's canal.

### FIELD OF THE INVENTION

The present invention relates to treating glaucoma in an eye of a subject.

### BACKGROUND

Traditional treatments for glaucoma include oral medication, eye drops, and surgery. Laser surgery, trabeculectomy, and drainage implant surgery are known treatment options. While these types of surgeries are often effective in lowering intraocular pressure they sometimes necessitate a scleral flap to be made in the eye, which can be a difficult procedure.

### SUMMARY OF THE INVENTION

In some applications of the present invention, glaucoma in an eye of a subject is treated by expanding the Schlemm's canal in the eye. Expanding the Schlemm's canal increases fluid flow into and/or within the Schlemm's canal, which effects a change in the fluid permeability of the surrounding tissue, known as the trabecular meshwork, and increases fluid outflow, through native valves, that has been disrupted by the glaucoma, thereby lowering intraocular pressure.

A method is provided for locating the Schlemm's canal within an eye of a subject by introducing a visible dye into the eye such that the dye flows into the Schlemm's canal, and subsequently observing the location of the dye. Additional methods of localization may use various combinations of medical imagining modalities, e.g., optical or ultrasonic. Once located, the Schlemm's canal can be expanded using one or more expandable porous structures. The expandable porous structures are introduced into the Schlemm's canal at discrete locations around the canal in a collapsed form, while disposed within a lumen of an introducer, and are configured to subsequently expand while inside the Schlemm's canal. The expandable structures may for example be expandable spherical, cylindrical, or ellipsoidal stents, configured to expand into their final shape inside the canal, or they may be in the form of a liquid hydrogel, configured to be crosslinked inside the Schlemm's canal.

There is therefore provided in accordance with an inventive concept 1 of the present invention, apparatus for expanding a Schlemm's canal of an eye of a subject, the apparatus including:
an introducer having a lumen; and
a plurality of expandable porous structures sized and shaped to be disposed within the lumen and configured (a) to be inserted into the Schlemm's canal at discrete locations around the canal, (b) to be inserted into the Schlemm's canal while in a collapsed form, and (c) to expand while inside the Schlemm's canal.
Inventive concept 2. The apparatus according to inventive concept 1, wherein the expandable porous structures are shaped to define pores having an average pore size of 5-50 microns.
Inventive concept 3. The apparatus according to inventive concept 1, wherein a length of each expandable porous structure, when expanded and when not constrained by any external force, is 50-200 microns.
Inventive concept 4. The apparatus according to inventive concept 1, wherein the expandable porous structures are ceramic structures.
Inventive concept 5. The apparatus according to inventive concept 1, wherein the expandable porous structures are polymer structures.
Inventive concept 6. The apparatus according to inventive concept 1, further including:
   a first longitudinal element, configured to protrude from a distal end of the introducer upon the expandable porous structure in collapsed form being pushed out of the lumen, and configured to subsequently retract proximally into the lumen; and
   a second longitudinal element,
   wherein the first and second longitudinal elements are disposed such that:
      a distal end of the second longitudinal element is coupled to a distal end of the expandable porous structure, and
      a proximal end of the second longitudinal element is coupled to a distal end of the first longitudinal element, and
   wherein the second longitudinal element is configured to expand the expandable porous structure in collapsed form into an expanded porous structure, upon retraction of the first longitudinal element into the lumen.
Inventive concept 7. The apparatus according to inventive concept 1, further including a deflated balloon, the balloon (a) disposed within the expandable porous structure in collapsed form, (b) coupled to a distal end of the introducer, and (c) configured to expand the expandable porous structure in collapsed form into an expanded porous structure by being inflated while inside the expandable porous structure in collapsed form.
Inventive concept 8. The apparatus according to inventive concept 1, wherein each of the expandable porous structures in collapsed form is under constraint while inside the lumen and is configured to automatically expand upon removal of the constraint by the expandable porous structure being pushed out of the lumen.
Inventive concept 9. The apparatus according to any one of inventive concepts 1-3 and 6-8, wherein the expandable porous structures are cylindrical structures.
Inventive concept 10. The apparatus according to inventive concept 9, wherein the cylindrical structures are cylindrical metal structures.
Inventive concept 11. The apparatus according to any one of inventive concepts 1-3 and 6-8, wherein the expandable porous structures are ellipsoidal structures.
Inventive concept 12. The apparatus according to inventive concept 11, wherein the ellipsoidal structures are ellipsoidal metal structures.
Inventive concept 13. The apparatus according to any one of inventive concepts 1-3 and 6-8, wherein the expandable porous structures are spherical structures.
Inventive concept 14. The apparatus according to inventive concept 13, wherein the spherical structures are spherical metal structures.
Inventive concept 15. The apparatus according to any one of inventive concepts 1-8, further including a light source, configured to emit light to locate the Schlemm's canal.
Inventive concept 16. The apparatus according to inventive concept 15, wherein the light source is configured to emit the light at a wavelength of 400-700 nm.
Inventive concept 17. The apparatus according to inventive concept 15, wherein the light source is configured to cause fluorescence of a fluorescent dye in the Schlemm's canal by emitting the light.
Inventive concept 18. The apparatus according to inventive concept 17, wherein the light source is configured to cause the fluorescence by emitting the light at a wavelength of 450-500 nm.
Inventive concept 19. The apparatus according to inventive concept 17, wherein the light source is configured to emit the light that causes the fluorescence as ultraviolet (UV) light.
Inventive concept 20. The apparatus according to inventive concept 19, wherein the light source is configured to cause the fluorescence by emitting the UV light at a wavelength of 250-400 nm.
Inventive concept 21. The apparatus according to inventive concept 17, wherein the apparatus further includes the fluorescent dye.
Inventive concept 22. The apparatus according to inventive concept 17, wherein the apparatus further includes:
   a camera, configured to obtain data corresponding to a location of the fluorescent dye in the Schlemm's canal;
   a mechanical actuator; and
   control circuitry coupled to the mechanical actuator,
      wherein the camera is configured to send the data to the control circuitry, and the control circuitry is configured to activate the mechanical actuator to advance the introducer toward the location in the Schlemm's canal.
Inventive concept 23. The apparatus according to inventive concept 17, wherein the apparatus further includes:
   a mount, configured to hold the introducer at a predetermined angle with respect to the eye; and
   control circuitry coupled to the mount,
      wherein the control circuitry is configured to (a) obtain data corresponding to a location of the fluorescent dye in the Schlemm's canal, (b) determine the angle, with respect to the eye, at which the mount is required to hold the introducer such that a trajectory from the introducer to the eye intersects the eye at the location of the fluorescent dye, and (c) activate the mount to orient itself such that the introducer is disposed at the determined angle with respect to the eye.
Inventive concept 24. The apparatus according to any one of inventive concepts 1-3, wherein each of the expandable porous structures is a hydrogel, wherein the introducer is an injector, configured to inject the hydrogel in the form of a liquid bolus into the Schlemm's canal.
Inventive concept 25. The apparatus according to inventive concept 24, wherein the apparatus includes a light source, configured to emit light that crosslinks the hydrogel.
Inventive concept 26. The apparatus according to inventive concept 25, wherein the light source is configured to emit the light as infrared (IR) light.
Inventive concept 27. The apparatus according to inventive concept 26, wherein the light source is configured to emit the IR light at a wavelength of 700 nm-25 microns.
Inventive concept 28. The apparatus according to inventive concept 26, wherein the light source is configured to emit the IR light as near-infrared (NIR) light.
Inventive concept 29. The apparatus according to inventive concept 26, wherein the light source is configured to emit the IR light as far-infrared (FIR) light.
Inventive concept 30. The apparatus according to inventive concept 25, wherein the light source is configured to emit the light at a wavelength of 400-700 nm.
Inventive concept 31. The apparatus according to inventive concept 25, wherein the light source is configured to emit the light as ultraviolet (UV) light.
Inventive concept 32. The apparatus according to inventive concept 31, wherein the light source is configured to emit the UV light at a wavelength of 250-400 nm.
Inventive concept 33. The apparatus according to inventive concept 25, wherein the injector includes the light source.
Inventive concept 34. The apparatus according to inventive concept 33, wherein the injector includes (a) a tube, and (b) a plunger sized and shaped to be slidably advanceable within the tube, and wherein the light source is configured to emit the light that crosslinks the hydrogel upon advancement of the plunger within the tube.

Inventive concept 35. The apparatus according to claim 34, wherein the light source is configured to emit the light that crosslinks the hydrogel upon the hydrogel being fully injected into the Schlemm's canal.
Inventive concept 36. The apparatus according to inventive concept 33, wherein the light source is configured to emit the light as infrared (IR) light.
Inventive concept 37. The apparatus according to inventive concept 36, wherein the light source is configured to emit the IR light at a wavelength of 700 nm-25 microns.
Inventive concept 38. The apparatus according to inventive concept 36, wherein the light source is configured to emit the IR light as near-infrared (NIR) light.
Inventive concept 39. The apparatus according to inventive concept 36, wherein the light source is configured to emit the IR light as far-infrared (FIR) light.
Inventive concept 40. The apparatus according to inventive concept 33, wherein the light source is configured to emit the light at a wavelength of 400-700 nm.
Inventive concept 41. The apparatus according to inventive concept 33, wherein the light source is configured to emit the light as ultraviolet (UV) light.
Inventive concept 42. The apparatus according to inventive concept 41, wherein the light source is configured to emit the UV light at a wavelength of 250-400 nm.
Inventive concept 43. The apparatus according to inventive concept 33, wherein the light source is configured to also emit light to locate the Schlemm's canal.
Inventive concept 44. The apparatus according to inventive concept 43, wherein the light source is configured to emit the light to locate the Schlemm's canal at a wavelength of 400-700 nm.
Inventive concept 45. The apparatus according to inventive concept 43, wherein the light source is configured to cause fluorescence of a fluorescent dye in the Schlemm's canal by emitting the light.
Inventive concept 46. The apparatus according to inventive concept 45, wherein the light source is configured to cause the fluorescence by emitting the light at a wavelength of 450-500 nm.
Inventive concept 47. The apparatus according to inventive concept 45, wherein the light source is configured to emit the light that causes the fluorescence as ultraviolet (UV) light.
Inventive concept 48. The apparatus according to inventive concept 47, wherein the light source is configured to cause the fluorescence by emitting the UV light at a wavelength of 250-400 nm.
Inventive concept 49. The apparatus according to inventive concept 45, wherein the light source is configured to emit the light for causing fluorescence of a fluorescent dye at a first wavelength, and to emit the light for crosslinking the hydrogel at a second wavelength.
Inventive concept 50. The apparatus according to inventive concept 49, wherein the first wavelength is not equal to the second wavelength.
Inventive concept 51. The apparatus according to inventive concept 49, wherein the first wavelength is equal to the second wavelength.
Inventive concept 52. The apparatus according to inventive concept 51, wherein:
   the light source is configured to emit the light for causing fluorescence of the fluorescent dye at a first energy,
   the light source is configured to emit the light for crosslinking the hydrogel at a second energy, and
   the second energy is higher than the first energy.
Inventive concept 53. The apparatus according to inventive concept 24, wherein the injector is configured to inject the hydrogel bolus into the Schlemm's canal in a dehydrated form that is expandable due to hydration upon contact with fluids within the Schlemm's canal.
Inventive concept 54. The apparatus according to inventive concept 53, wherein the injector is configured to further inject a fluid to expand the dehydrated hydrogel into a hydrated state within the Schlemm's canal.
Inventive concept 55. The apparatus according to any one of inventive concepts 1-3, wherein each of the expandable porous structures is a cryogel, wherein the introducer is an injector, configured to inject the cryogel into the Schlemm's canal in a freeze-dried form that is expandable due to hydration upon contact with fluids within the Schlemm's canal.
Inventive concept 56. The apparatus according to inventive concept 55, wherein the injector is configured to further inject a fluid to expand the cryogel into a hydrated state within the Schlemm's canal.

There is further provided, in accordance with an inventive concept 57 of the present invention, a method for locating a Schlemm's canal of an eye of a subject, the method including:
introducing a visible dye into the eye such that the dye flows into the Schlemm's canal;
subsequently, locating the Schlemm's canal by observing the dye; and
treating the Schlemm's canal based on the locating.
Inventive concept 58. The method according to inventive concept 57, wherein locating the Schlemm's canal includes exposing the eye to light having a wavelength of 400-700 nm.
Inventive concept 59. The method according to inventive concept 57, wherein introducing the visible dye includes injecting the dye into a vitreous body of the eye.
Inventive concept 60. The method according to inventive concept 57, wherein introducing the visible dye includes placing drops onto an outer surface of the eye.
Inventive concept 61. The method according to inventive concept 57, wherein introducing the visible dye includes injecting the dye into a vein that drains from the Schlemm's canal.
Inventive concept 62. The method according to inventive concept 61, wherein injecting the dye into the vein includes injecting the dye into a visible aqueous vein around the Schlemm's canal.
Inventive concept 63. The method according to inventive concept 61, wherein injecting the dye into the vein includes injecting the dye into an episcleral vein that leads from a visible aqueous vein around the Schlemm's canal.
Inventive concept 64. The method according to any one of inventive concepts 57-63, wherein the dye is a fluorescent dye, and further including causing the dye to fluoresce.
Inventive concept 65. The method according to inventive concept 64, wherein causing the dye to fluoresce includes exposing the eye to light having a wavelength of 450-500 nm.
Inventive concept 66. The method according to inventive concept 64, wherein causing the dye to fluoresce includes exposing the eye to ultraviolet (UV) light.
Inventive concept 67. The method according to inventive concept 66, wherein exposing the eye to the UV light includes exposing the eye to UV light having a wavelength of 250-400 nm.

There is further provided, in accordance with an inventive concept 68 of the present invention, a method for expanding a Schlemm's canal of an eye of a subject, the method including:
inserting into the Schlemm's canal a plurality of expandable porous structures at discrete locations around the canal, while the expandable porous structures are in collapsed form; and
causing the expandable porous structures in collapsed form to expand while inside the Schlemm's canal.
Inventive concept 69. The method according to inventive concept 68, wherein causing the expandable porous structures in collapsed form to expand includes causing each expandable porous structure in collapsed form to expand prior to inserting a subsequent one of the expandable porous structures.
Inventive concept 70. The method according to inventive concept 68, wherein inserting includes inserting the plurality of expandable porous structures into the Schlemm's canal at discrete locations in the Schlemm's canal that are not locations of visible Schlemm's canal valves.
Inventive concept 71. The method according to inventive concept 68, wherein inserting includes inserting the plurality of expandable porous structures into the Schlemm's canal at discrete locations in the Schlemm's canal that are at least 10 degrees apart along the circumference of the Schlemm's canal.
Inventive concept 72. The method according to inventive concept 68, wherein inserting includes inserting the plurality of expandable porous structures into the Schlemm's canal at discrete locations in the Schlemm's canal that are evenly spaced around the Schlemm's canal.
Inventive concept 73. The method according to inventive concept 68, wherein inserting includes inserting a first expandable porous structure into the Schlemm's canal at a first location, and subsequently inserting a second expandable porous structure into the Schlemm's canal at a second location, wherein the second location is greater than 90 degrees away from the first location.
Inventive concept 74. The method according to inventive concept 68, wherein inserting includes inserting a first expandable porous structure into the Schlemm's canal at a first location, inserting a second expandable porous structure into the Schlemm's canal at a second location, wherein the second location is between 150 and 210 degrees away from the first location, and subsequently inserting a third expandable porous structure in the Schlemm's canal at a third location, wherein the third location is greater than 45 degrees away from the first and second locations.
Inventive concept 75. The method according to inventive concept 68-74, wherein inserting includes:
   inserting each of the plurality of expandable porous structures into the Schlemm's canal while the expandable porous structure in collapsed form is disposed within a lumen of an introducer; and
   subsequently pushing the expandable porous structure in collapsed form out of the lumen and into the Schlemm's canal.
Inventive concept 76. The method according to inventive concept 75, wherein causing the expandable porous structure to expand includes pushing the expandable porous structure in collapsed form out of the lumen, wherein the expandable porous structure in collapsed form is under constraint while inside the lumen and is configured to naturally expand upon removal of the constraint by pushing the expandable porous structure out of the lumen.
Inventive concept 77. The method according to inventive concept 75, wherein causing the expandable porous structure in collapsed form to expand includes retracting, proximally into a distal end of the introducer, a first longitudinal element, wherein:
   (a) subsequent to the expandable porous structure in collapsed form being pushed out of the lumen, the first longitudinal element protrudes from a distal end of the introducer, and
   (b) a distal end of the first longitudinal element is coupled to a proximal end of a second longitudinal element, wherein a distal end of the second longitudinal element is coupled to a distal end of the expandable porous structure.
Inventive concept 78. The method according to inventive concept 75, wherein causing the expandable porous structure in collapsed form to expand includes inflating a balloon inside the expandable porous structure in collapsed form.
Inventive concept 79. The method according to inventive concept 68-74, wherein inserting includes:
   using an injector to inject into the Schlemm's canal a hydrogel in the form of a liquid bolus; and
   crosslinking the hydrogel.
Inventive concept 80. The method according to inventive concept 79, wherein crosslinking the hydrogel includes applying convection heat to the hydrogel.
Inventive concept 81. The method according to inventive concept 79, wherein crosslinking the hydrogel includes exposing the hydrogel to light that crosslinks the hydrogel.
Inventive concept 82. The method according to inventive concept 81, wherein exposing the hydrogel to the light includes exposing the hydrogel to infrared (IR) light.
Inventive concept 83. The method according to inventive concept 82, wherein exposing the hydrogel to the IR light includes exposing the hydrogel to IR light having a wavelength of 700 nm to 25 microns.
Inventive concept 84. The method according to inventive concept 82, wherein exposing the hydrogel to the IR light includes exposing the hydrogel to near-infrared (NIR) light.
Inventive concept 85. The method according to inventive concept 82, wherein exposing the hydrogel to the IR light includes exposing the hydrogel to far-infrared (FIR) light.
Inventive concept 86. The method according to inventive concept 81, wherein exposing the hydrogel to the light includes exposing the hydrogel to light having a wavelength of 400-700 nm.
Inventive concept 87. The method according to inventive concept 86, wherein exposing the hydrogel to the light includes exposing the hydrogel to light having a wavelength of 450-500 nm, that causes fluorescence of a fluorescent dye in the Schlemm's canal.
Inventive concept 88. The method according to inventive concept 87, wherein exposing the hydrogel to the light includes:
   exposing the eye to the light that causes fluorescence of a fluorescent dye in the Schlemm's canal at a first energy; and
   subsequently, exposing the hydrogel to the light that crosslinks the hydrogel at a second energy,
   wherein the second energy is higher than the first energy.
Inventive concept 89. The method according to inventive concept 81, wherein exposing the hydrogel to the light includes exposing the hydrogel to ultraviolet (UV) light.
Inventive concept 90. The method according to inventive concept 89, wherein exposing the hydrogel to the UV light includes exposing the hydrogel to UV light having a wavelength of 250-400 nm.
Inventive concept 91. The method according to inventive concept 89, wherein exposing the hydrogel to the UV light includes exposing the hydrogel to UV light that causes fluorescence of a fluorescent dye in the Schlemm's canal.
Inventive concept 92. The method according to inventive concept 91, wherein exposing the hydrogel to the UV light includes:
   exposing the eye to the UV light that causes fluorescence of a fluorescent dye in the Schlemm's canal at a first energy; and
   subsequently, exposing the hydrogel to the UV radiation that crosslinks the hydrogel at a second energy,
   wherein the second energy is higher than the first energy.
Inventive concept 93. The method according to inventive concept 81, wherein exposing the hydrogel to light includes activating a light source coupled to the injector.
Inventive concept 94. The method according to inventive concept 93, wherein activating includes advancing a plunger into a tube of the injector.
Inventive concept 95. The method according to inventive concept 79, wherein injecting includes injecting the hydrogel into the Schlemm's canal in a dehydrated form.
Inventive concept 96. The method according to inventive concept 95, wherein injecting includes injecting the hydrogel into the Schlemm's canal in a dehydrated form, and subsequently actively hydrating the dehydrated hydrogel.
Inventive concept 97. The method according to inventive concept 96, wherein actively hydrating includes further injecting a fluid into the dehydrated hydrogel within the Schlemm's canal.
Inventive concept 98. The method according to inventive concept 68-74, wherein inserting includes:
   using an injector to inject into the Schlemm's canal a cryogel in the form of a freeze-dried bolus; and
   hydrating the cryogel.
Inventive concept 99. The method according to inventive concept 98, wherein hydrating further includes injecting a fluid into the cryogel within the Schlemm's canal.

The present invention will be more fully understood from the following detailed description of applications thereof, taken together with the drawings, in which:

### BRIEF DESCRIPTION OF THE DRAWINGS

FIGS. 1A-B are schematic illustrations of a dye being injected into an eye of a subject, and the eye being exposed to an external light source, in accordance with some applications of the present invention;

FIGS. 2A-B are schematic illustrations of a dye being injected into a vein in the eye, and the eye being exposed to an external light source, in accordance with some applications of the present invention;

FIGS. 3A-B are schematic illustrations of drops being placed into the eye of the subject, and the eye being exposed to an external light source, in accordance with some applications of the present invention;

FIG. 4A is a schematic illustration of an expandable structure inside an introducer tube, in accordance with some applications of the present invention;

FIGS. 4B-C are schematic illustrations of the expandable porous structure of FIG. 4A being pushed out of the introducer tube and subsequently expanded, in accordance with some applications of the present invention;

FIG. 5A is a schematic illustration of an expandable structure inside an introducer tube, in accordance with some applications of the present invention;

FIGS. 5B-D are schematic illustrations of the expandable structure of FIG. 5A being pushed out of the introducer tube and subsequently expanded, in accordance with some applications of the present invention;

FIGS. 6A-B are schematic illustrations of a liquid hydrogel being injected into a Schlemm's canal of the eye of the subject, and the eye being exposed to an external light source, in accordance with some applications of the present invention;

FIGS. 7A-B are schematic illustrations of a liquid hydrogel being injected into the Schlemm's canal of the eye, and the eye being exposed to a light source disposed on the injector, in accordance with some applications of the present invention; and

FIG. 8 is a schematic illustration of the eye with expandable structures distributed around the Schlemm's canal, in accordance with some applications of the present invention.

### DETAILED DESCRIPTION

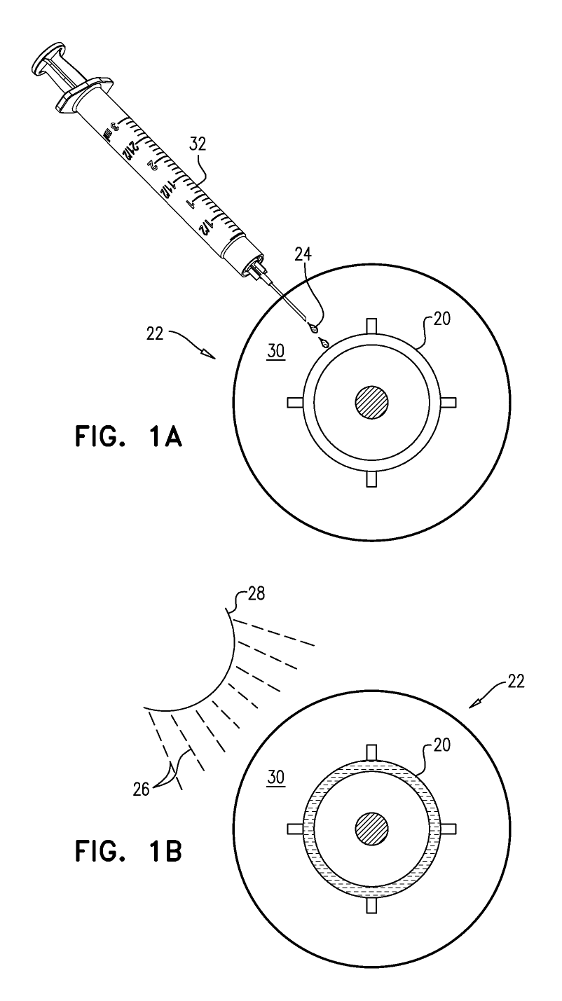

Reference is made to FIGS. 1A-B, 2A-B, and 3A-B, which are schematic illustrations of a method used to locate a Schlemm's canal 20 in an eye 22 of a subject, in accordance with some applications of the present invention. In some applications, a visible dye 24 is introduced into eye 22 such that dye 24 flows into Schlemm's canal 20. Schlemm's canal 20 can subsequently be located by observing visible dye 24. Typically, visible dye 24 is visible in Schlemm's canal 20 when eye 22 is exposed to light 26, emitted from a light source 28.

In some applications, dye 24 is a fluorescent dye and light source 28 is configured to emit light 26 in order to cause fluorescent dye 24 to fluoresce, thereby making Schlemm's canal 20 visible. Light 26 that is used to locate Schlemm's canal 20 by causing fluorescence of fluorescent dye in Schlemm's canal 20 may be, for example, broadband or narrowband light, e.g., visible light (at a wavelength of 400-700 nm, e.g., 450-500 nm) or UV light (e.g., at a wavelength of 250-400 nm).

In some applications, dye 24 is injected into a vitreous body 30 of eye 22, using an injector 32 (FIG. 1A). Visible dye 24 flows from vitreous body 30 into Schlemm's canal 20, facilitating the ability to locate Schlemm's canal 20 when eye 22 is exposed to light 26 emitted from light source 28 (FIG. 1B).

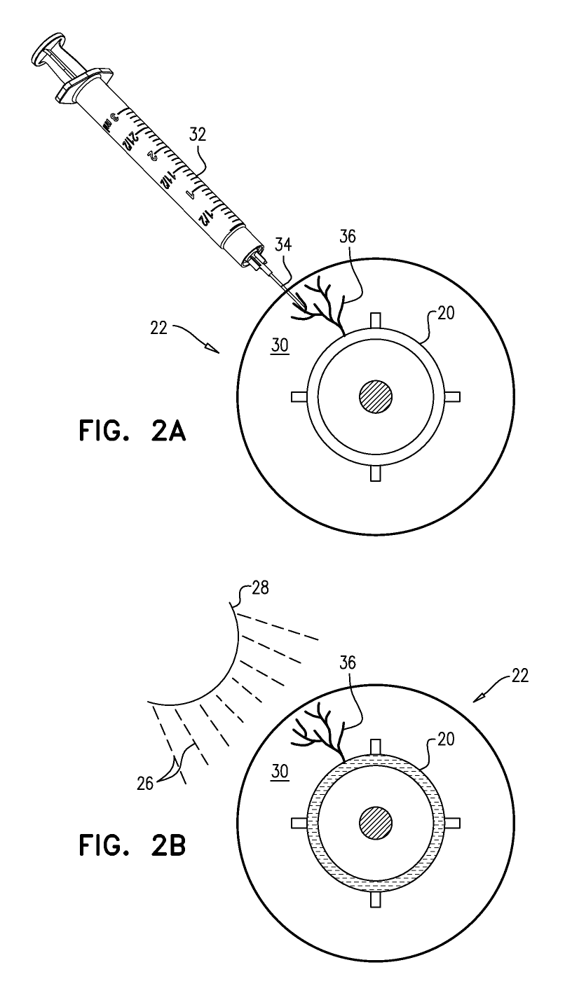

In some applications, a fine syringe 34 is used to inject dye 24 into a vein 36 that drains from Schlemm's canal 20 (FIG. 2A). Typically, vein 36 is either (a) any visible aqueous vein around Schlemm's canal 20 or (b) an episcleral vein that leads from the aqueous veins. Once inside Schlemm's canal 20, dye 24 facilitates the ability to locate Schlemm's canal 20 when eye 22 is exposed to light 26 emitted from light source 28 (FIG. 2B).

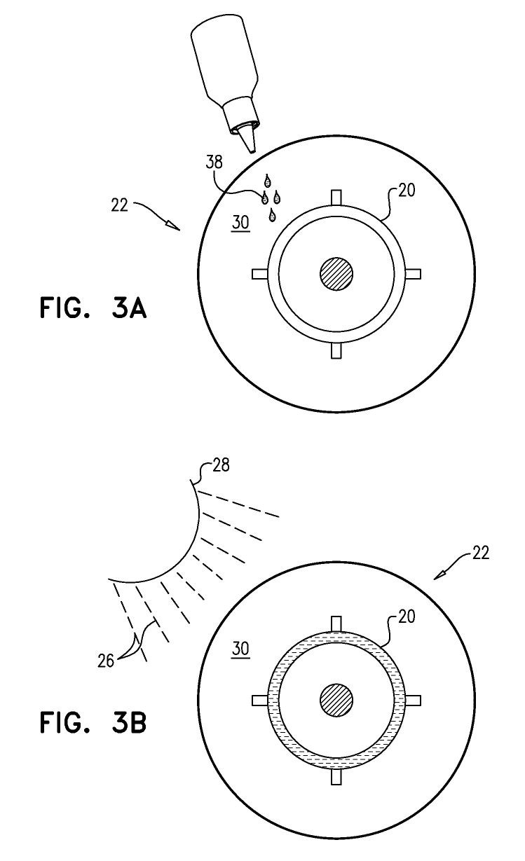

In some applications, dye 24 is introduced into eye 22 in the form of eye drops 38 which comprise dye 24 (FIG. 3A). Upon drops 38 entering Schlemm's canal 20, drops 38 encounter a different diffusion and uptake mechanism than in tissue surrounding Schlemm's canal 20, such that inside Schlemm's canal 20 drops 38 maintain a higher concentration of dye 24, facilitating the ability to locate Schlemm's canal 20 when eye 22 is exposed to light 26 emitted from light source 28 (FIG. 3B)

In other applications, due to Schlemm's canal 20 having optical properties that differ from the optical properties of surrounding tissue, Schlemm's canal 20 can be located by exposing eye 22 to broadband or narrowband light, e.g., visible light in the range of 400-700 nm. Alternatively, Schlemm's canal 20 can be located using one or more medical imaging techniques, such as gonioscopy, ultrasound biomicroscopy, or optical coherence tomography. For example, ultrasound imaging may be used to observe the phonic properties of Schlemm's canal 20, in order to provide adequate contrast to identify the canal.

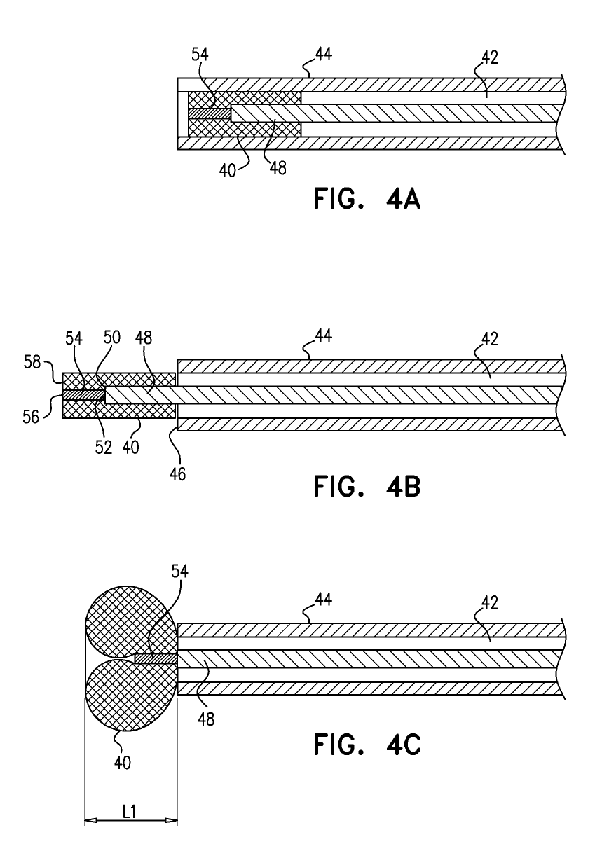

Reference is now made to FIGS. 4A-C, which are schematic illustrations of apparatus for expanding Schlemm's canal 20, in accordance with some applications of the present invention. In some applications, a plurality of expandable porous structures 40 are used to expand Schlemm's canal 20 in eye 22. Expandable porous structures 40 are inserted into Schlemm's canal 20 while in a collapsed form and subsequently expanded while inside Schlemm's canal 20. Typically, each expandable porous structure 40 is sized and shaped to be disposed in a lumen 42 of an introducer 44 while in collapsed form (FIG. 4A). Once expanded, expandable porous structures 40 may have various geometries, e.g., expandable porous structures 40 may be spherical structures, cylindrical structures, or ellipsoidal structures. Typically, expandable porous structures 40 are either metal, polymer, or ceramic. Typically, when expanded, (a) expandable porous structures 40 are shaped to define pores having an average pore size of 5-microns, and (b) a length L1 of each expandable porous structure 40, when not constrained by any external force, is 50-200 microns.

In some applications, a series of longitudinal elements coupled to expandable porous structure 40 and introducer 44 are used to expand each expandable porous structure 40 inside Schlemm's canal 20 (FIG. 4A). Once each expandable porous structure 40 is pushed out of a distal end 46 of introducer 44 in collapsed form, a first longitudinal element 48 protrudes from distal end 46 of introducer 44 and is configured to retract proximally into lumen 42 of introducer 44 (FIG. 4B). A distal end 50 of first longitudinal element 48 is coupled to a proximal end 52 of a second longitudinal element 54, and a distal end 56 of second longitudinal element 54 is coupled to a distal end 58 of expandable porous structure 40 in collapsed form. When first longitudinal element 48 is retracted proximally into lumen 42, second longitudinal element 54 is pulled proximally as well, causing expandable porous structures 40 in collapsed form to expand (FIG. 4C).

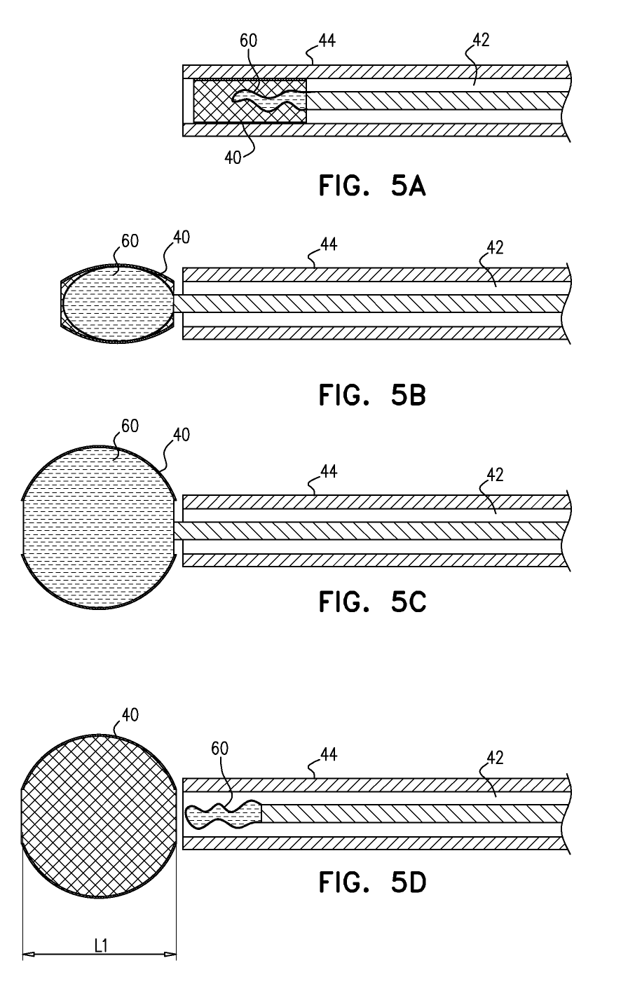

Reference is now made to FIGS. 5A-D, which are schematic illustrations of apparatus for expanding Schlemm's canal 20, in accordance with some applications of the present invention. In some applications, a deflated balloon 60, disposed inside each expandable porous structure 40 (FIG. 5A), is used to expand each expandable porous structure 40 inside Schlemm's canal 20. Each expandable porous structure 40 in collapsed form is pushed out of lumen 42 inside Schlemm's canal 20 (FIG. 5B), and subsequently balloon 60 is inflated, causing expandable porous structures 40 to expand (FIG. 5C). Balloon may then be deflated and removed from expanded porous structure 40 (FIG. 5D).

In some applications, each expandable porous structure 40 in collapsed form is under constraint while inside lumen 42 of introducer 44, and is configured to automatically expand upon removal of the constraint, such that upon being pushed out of introducer 44 into Schlemm's canal 20, expandable porous structure 40 automatically expands on its own without anything being actively done by the surgeon to cause the expansion.

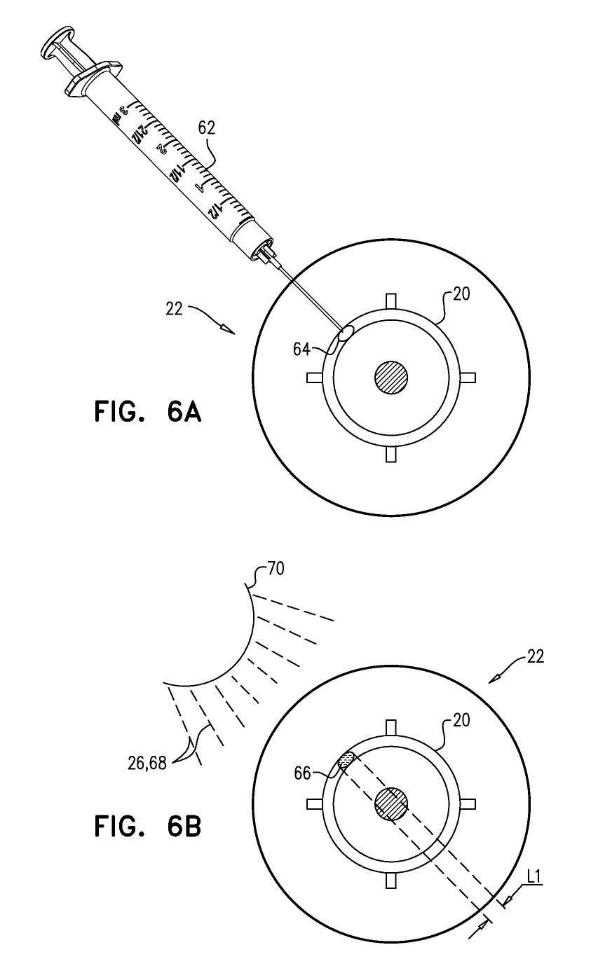

Reference is now made to FIGS. 6A-B, which are schematic illustrations of apparatus for expanding Schlemm's canal 20, in accordance with some applications of the present invention. In some applications, each expandable porous structure 40 is a hydrogel. Using an injector 62, the hydrogel is injected into Schlemm's canal 20 in the form of a liquid bolus 64 (FIG. 6A). The hydrogel is subsequently crosslinked inside Schlemm's canal 20 to become a crosslinked hydrogel structure 66 which locally expands Schlemm's canal 20 (FIG. 6B).

Typically, hydrogel bolus 64, inside Schlemm's canal 20, is crosslinked by exposing it to light 68, emitted from a light source 70. Examples of light 68 that can be used are infrared (IR) light (at a wavelength of 700 nm to 25 microns), near-infrared (NIR) light (700-2500 nm), far-infrared (FIR) light (15 microns-1 mm), visible light (at a wavelength of 400-700 nm), and ultraviolet (UV) light (e.g., at a wavelength of 250-400 nm).

In some applications, light source 70 is configured to emit light 68, used to crosslink hydrogel bolus 64, as well as light 26, used to locate Schlemm's canal 20 (FIGS. 1A-B). For some applications, the wavelength of light 68, used to crosslink hydrogel bolus 64, is different from the wavelength of light 26, used to locate Schlemm's canal 20 (e.g., at least 50 nm apart). For some applications, the wavelength of light 68, used to crosslink hydrogel bolus 64, is the same as the wavelength of light 26, used to locate Schlemm's canal 20. In such a case, light 68 and light 26 may be emitted at the same or different respective energies. For example, Schlemm's canal 20 may first be located by exposing eye 22 to light 26 at a first energy, and subsequent to hydrogel bolus 64 being injected into Schlemm's canal 20, eye 22 is exposed to light 68 at a second energy, to crosslink the hydrogel. Typically, the second energy is higher than the first energy.

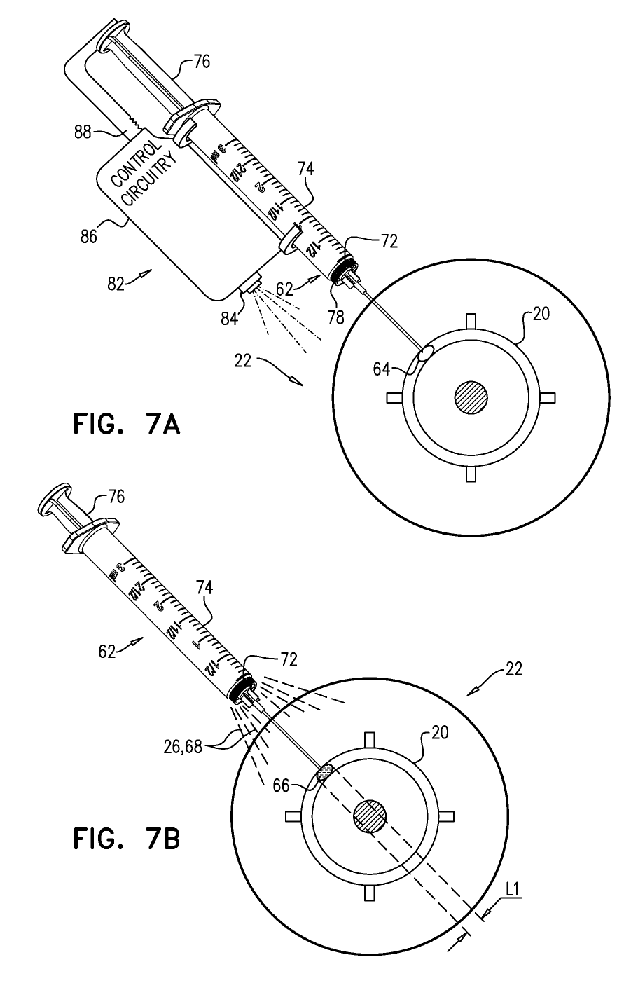

Reference is now made to FIGS. 7A-B, which are schematic illustrations of apparatus for expanding Schlemm's canal 20, in accordance with some applications of the present invention. In some applications, injector 62 has a tube 74, a plunger 76, sized and shaped to be slidably advanceable within tube 74, and a light source 72, disposed at a distal end 78 of tube 74. Injector 62 is configured to emit light 68 from light source 72 upon advancement of plunger 76 within tube 74, such that light source 72 is automatically activated to emit light 68 without necessitating a switch in instruments or a break to activate an external light source. In some applications, injector 62 is configured to emit light 68 upon hydrogel bolus 64 being fully injected into Schlemm's canal 20.

In some applications, injector 62 is configured to emit light 26 used to locate Schlemm's canal 20 (FIG. 1B) as well as light 68 used to crosslink hydrogel bolus 64. Light 26 that is used to locate Schlemm's canal 20 by causing fluorescence of fluorescent dye 24 in Schlemm's canal 20 may be, for example, broadband or narrowband light, e.g., visible light (at a wavelength of 400-700 nm, e.g., 450-500 nm) or UV light (e.g., at a wavelength of 250-400 nm). Light 68 that is used for crosslinking may be, for example, IR light (at a wavelength of 700 nm-25 microns), NIR light (700-2500 nm), FIR light (15 microns-1 mm), visible light (at a wavelength of 400-700 nm), or UV light (e.g., at a wavelength of 250-400 nm). In some applications, the wavelength of light 26, used to cause fluorescence, is the same as the wavelength of light 68, used for crosslinking the hydrogel, in which case, injector 62 is configured to emit light 26 at a first energy and to emit light 68 at a second energy. Typically, the second energy is higher than the first energy.

In some applications, hydrogel bolus 64 is crosslinked by applying convection heat to eye 22, e.g., 25-100 degrees Celsius. In other applications, hydrogel bolus 64 is given time to crosslink on its own, in which case nothing is actively done by the surgeon to crosslink the hydrogel.

In some applications, hydrogel bolus 64 is implanted while in a dry or dehydrated form and is subsequently expanded by hydration upon contact with fluids within Schlemm's canal 20 or upon an additional injection from injector 62. In other applications, hydrogel bolus 64 is a cryogel or a freeze-dried soluble material that is expanded by hydration upon contact with fluids within Schlemm's canal 20 or upon an additional injection from injector 62.

Typically, but not necessarily, the insertion of each hydrogel bolus 64, as shown in FIG. 7A, may be implemented with a control system 82. For example, a camera 84 may be used to obtain data corresponding to a location of fluorescent dye 24 in Schlemm's canal 20. Camera 84 then sends the data to control circuitry 86, which is coupled to a mechanical actuator 88. Control circuitry 86 activates mechanical actuator 88 to advance injector 62 toward the location in Schlemm's canal 20. Control system 82 may, similarly, be used for the insertion of expandable porous structures 40 as they are shown in FIGS. 4A-C and FIGS. 5A-D.

In one application of injecting the hydrogel, the surgeon has a tool in the form of a gimbal mount which is placed above eye 22. The gimbal mount can rotate with three degrees of freedom around an axis largely collinear with, or largely parallel to, the central axis of eye 22 formed between the centroid point of the cornea and the centroid point of the retina. Thereafter, following the identification of Schlemm's canal 20 using any of the methods previously mentioned, a targeting method is applied that creates the trajectory from the gimbal mount to Schlemm's canal 20. The targeting method may be line of sight for the surgeon, a camera, a laser pointer, or a combination thereof, to provide and fix a well-defined trajectory emanating from the gimbal mount to Schlemm's canal 20. Once the trajectory is fixed, the surgeon is able to insert injector 62 by following the trajectory, and enter Schlemm's canal 20 through exterior tissues of eye 22, whether they be sclera, cornea, or corneoscleral junction. The gimbal mount, as described hereinabove, may similarly be used for the insertion of expandable porous structures 40 as they are shown in FIGS. 4A-C and FIGS. 5A-D.

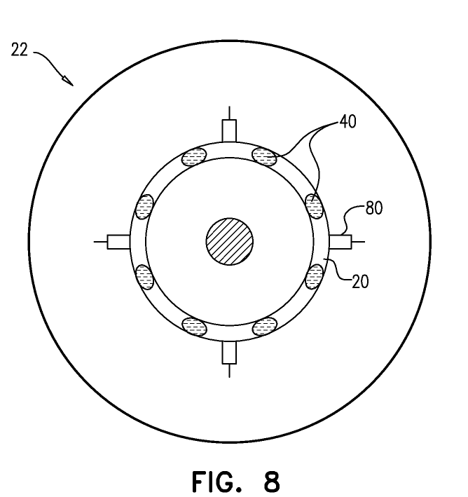

Reference is now made to FIG. 8, which is a schematic illustration of apparatus for expanding Schlemm's canal 20, in accordance with some applications of the present invention. Schlemm's canal 20 has a plurality of valves 80 located at discrete locations around the canal. Typically, the surgeon inserts expandable porous structures 40 into Schlemm's canal at one or more evenly-spaced discrete locations in Schlemm's canal 20, while making sure to avoid the locations of any visible Schlemm's canal valves 80.

In some applications, the surgeon achieves generally even spacing of expandable porous structures 40 in Schlemm's canal 20 by inserting the first two expandable porous structures 40 at two different locations that are substantially opposite each other (e.g., 150-210 degrees), subsequently inserting a third expandable porous structure 40 at a third location that is at least 45 degrees away from the first two locations, and continuing this process of inserting each next expandable porous structure 40 in-between two that have already been inserted. In some applications, the first two expandable porous structures 40 are inserted at two different locations that may not be substantially opposite each other, but are in any case greater than 90 degrees away from each other.

In some applications, for example, when light 68 for crosslinking the hydrogel is emitted from injector 62, each expandable porous structure 40, after being inserted in Schlemm's canal 20, is expanded prior to inserting the next expandable porous structure 40 into Schlemm's canal 20.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for expanding a Schlemm's canal of an eye of a subject, the apparatus comprising:
an introducer having a lumen; and
a plurality of expandable porous structures sized and shaped to be disposed within the lumen and configured (a) to be inserted into the Schlemm's canal at discrete locations around the canal, (b) to be inserted into the Schlemm's canal while in a collapsed form, and (c) to expand while inside the Schlemm's canal.

2. The apparatus according to claim 1, wherein the expandable porous structures are shaped to define pores having an average pore size of 5-50 microns.

3. The apparatus according to claim 1, wherein a length of each expandable porous structure, when expanded and when not constrained by any external force, is 50-200 microns.

4. The apparatus according to claim 1, wherein the expandable porous structures are polymer structures.

5. The apparatus according to claim 1, further comprising:
a first longitudinal element, configured to protrude from a distal end of the introducer upon the expandable porous structure in collapsed form being pushed out of the lumen, and configured to subsequently retract proximally into the lumen; and
a second longitudinal element,
wherein the first and second longitudinal elements are disposed such that:
a distal end of the second longitudinal element is coupled to a distal end of the expandable porous structure, and
a proximal end of the second longitudinal element is coupled to a distal end of the first longitudinal element, and
wherein the second longitudinal element is configured to expand the expandable porous structure in collapsed form into an expanded porous structure, upon retraction of the first longitudinal element into the lumen.

6. The apparatus according to claim 1, wherein each of the expandable porous structures in collapsed form is under constraint while inside the lumen and is configured to automatically expand upon removal of the constraint by the expandable porous structure being pushed out of the lumen.

7. The apparatus according to claim 1, wherein the expandable porous structures are cylindrical structures.

8. The apparatus according to claim 1, wherein the expandable porous structures are spherical structures.

9. The apparatus according to claim 1, further comprising a light source, configured to emit light to locate the Schlemm's canal.

10. The apparatus according to claim 9, wherein the light source is configured to cause fluorescence of a fluorescent dye in the Schlemm's canal by emitting the light.

11. The apparatus according to claim 10, wherein the apparatus further comprises:
a camera, configured to obtain data corresponding to a location of the fluorescent dye in the Schlemm's canal;
a mechanical actuator; and
control circuitry coupled to the mechanical actuator,
wherein the camera is configured to send the data to the control circuitry, and the control circuitry is configured to activate the mechanical actuator to advance the introducer toward the location in the Schlemm's canal.

12. The apparatus according to claim 10, wherein the apparatus further comprises:
a mount, configured to hold the introducer at a predetermined angle with respect to the eye; and
control circuitry coupled to the mount,
wherein the control circuitry is configured to (a) obtain data corresponding to a location of the fluorescent dye in the Schlemm's canal, (b) determine the angle, with respect to the eye, at which the mount is required to hold the introducer such that a trajectory from the introducer to the eye intersects the eye at the location of the fluorescent dye, and (c) activate the mount to orient itself such that the introducer is disposed at the determined angle with respect to the eye.

13. The apparatus according to claim 1, wherein each of the expandable porous structures is a hydrogel, wherein the introducer is an injector, configured to inject the hydrogel in the form of a liquid bolus into the Schlemm's canal.

14-16. (canceled)

17. A method for expanding a Schlemm's canal of an eye of a subject, the method comprising:
using (i) an introducer having a lumen and (ii) a plurality of expandable porous structures sized and shaped to be disposed within the lumen and configured (a) to be inserted into the Schlemm's canal at discrete locations around the canal, (b) to be inserted into the Schlemm's canal while in a collapsed form, and (c) to expand while inside the Schlemm's canal:
inserting into the Schlemm's canal the plurality of expandable porous structures at discrete locations around the canal, while the expandable porous structures are in collapsed form; and
causing the expandable porous structures in collapsed form to expand while inside the Schlemm's canal.

18. The method according to claim 17, wherein inserting comprises inserting the plurality of expandable porous structures into the Schlemm's canal at discrete locations in the Schlemm's canal that are not locations of visible Schlemm's canal valves.

19. The method according to claim 17, wherein inserting comprises inserting a first expandable porous structure into the Schlemm's canal at a first location, and subsequently inserting a second expandable porous structure into the Schlemm's canal at a second location, wherein the second location is greater than 90 degrees away from the first location.

20. The method according to claim 17, wherein:
using an introducer having a lumen comprises using an injector,
inserting comprises using the injector to inject into the Schlemm's canal a hydrogel in the form of a liquid bolus, and
the method further comprises crosslinking the hydrogel.
